(19) United States
(12) Patent Application Publication
(10) Pub. No.: US 2016/0031836 A1
(43) Pub. Date: Feb. 4, 2016

(54) BIOMARKER

(71) Applicants: Wenlai zhou, (US); Jinyun CHEN, (US); Christopher WILSON, (US); Yaoyu CHEN, (US)

(72) Inventors: Jinyun Chen, West Roxbury, MA (US); Yaoyu CHEN, Canton, MA (US); Christopher WILSON, Cambridge, MA (US); Wenlai ZHOU, Newton, MA (US)

(73) Assignee: Novartis AG, Basel (CH)

(21) Appl. No.: 14/774,511

(22) PCT Filed: Mar. 14, 2014
(86) PCT No.: PCT/IB2014/059826

§ 371 Date: Sep. 10, 2015

Related U.S. Application Data
(60) Provisional application No. 61/802,327, filed on Mar. 15, 2013.

(51) Int. Cl. C07D 261/18; C12Q 1/68

(52) U.S. Cl. CPC C07D 261/18; C12Q 1/6886; C12Q 2600/156; C12Q 2600/16; C12Q 2600/106; C12Q 2600/158

(57) ABSTRACT

The invention is directed, in part, to selective cancer treatment regimes based on assaying for the presence or absence of a mutation in a nucleic acid that encodes MLL1 or for the presence of reduced levels of MLL1.

### FIELD OF THE INVENTION

The disclosure is directed to novel personalized therapies, kits, transmittable forms of information and methods for use in treating patients having cancer.

### BACKGROUND OF THE INVENTION

Heat shock protein 90 (HSP90) is recognized as an anti-cancer target. Hsp90 is a highly abundant and essential protein which functions as a molecular chaperone to ensure the conformational stability, shape and function of client proteins. The Hsp90 family of chaperones is comprised of four members: Hsp90α and Hsp90β both located in the cytosol, GRP94 in the endoplasmic reticulum, and TRAP1 in the mitochondria. Hsp90 is an abundant cellular chaperone constituting about 1%-2% of total protein.

Among the stress proteins, Hsp90 is unique because it is not required for the biogenesis of most polypeptides. Hsp90 forms complexes with oncogenic proteins, called “client proteins”, which are conformationally labile signal transducers playing a critical role in growth control, cell survival and tissue development. Such binding prevents the degradation of these client proteins. A subset of Hsp90 client proteins, such as Raf, AKT, phospho-AKT, CDK4 and the EGFR family including ErbB2, are oncogenic signaling molecules critically involved in cell growth, differentiation and apoptosis, which are all processes important in cancer cells. Inhibition of the intrinsic ATPase activity of Hsp90 disrupts the Hsp90-client protein interaction resulting in their degradation via the ubiquitin proteasome pathway.

Hsp90 chaperones, which possess a conserved ATP-binding site at their N-terminal domain belong to a small ATPase sub-family known as the DNA Gyrase, Hsp90, Histidine Kinase and MutL (GHKL) sub-family. The chaperoning (folding) activity of Hsp90 depends on its ATPase activity which is weak for the isolated enzyme. However, it has been shown that the ATPase activity of Hsp90 is enhanced upon its association with proteins known as co-chaperones. Therefore, in vivo, Hsp90 proteins work as subunits of large, dynamic protein complexes. Hsp90 is essential for eukaryotic cell survival and is overexpressed in many tumors.

HSP90 inhibitors prevent the function of HSP90 assisting in the folding of nascent polypeptides and the correct assembly or disassembly of protein complexes and represses cancer cell growth, differentiation and survival. AUY922 and HSP990 are novel, non-geldanamycin-derivative HSP90 inhibitors and showed significant antitumor activities in a wide range of mutated and wild-type human cancer.

However, the efficacy of HSP90 inhibitors is decreased by cancer cell responses to HSP90 inhibition. Our previous study show that heat shock transcription factor1 (HSF1)-dependent heat shock response is important for mediating the positive feedback loop limiting the efficacy of HSP90 inhibitors. HSF1 knockdown combined with HSP90 inhibitors led to striking inhibitory effect on proliferation in vitro and tumor growth in vivo. HSF1 knockdown also enhanced the ability of HSP90 inhibitors to degrade oncogenic proteins, induce cancer cell apoptosis, and decrease activity of the ERK pathway. HSF1 expression is also significantly upregulated in HCC.

HSF1 transcriptional activities are induced by HSP90 inhibitors and provide a resistance mechanism through up-regulating a protective “heat shock” response and other transcriptional programs. However, HSF1 is a transcription factor and undruggable in current stage. This prompted us to identify critical druggable transcriptional modulators of HSF1 that are important for HSF1 transcriptional activities induced by HSP90 inhibitors. Those new identified HSF1− modulators will help us understand how HSF1 transcriptional function is regulated.

There is an increasing body of evidence that suggests a patient's genetic profile can be determinative to a patient's responsiveness to a therapeutic treatment. Given the numerous therapies available to an individual having cancer, a determination of the genetic factors that influence, for example, response to a particular drug, could be used to provide a patient with a personalized treatment regime. Such personalized treatment regimes offer the potential to maximize therapeutic benefit to the patient while minimizing related side effects that can be associated with alternative and less effective treatment regimes. Thus, there is a need to identify factors which can be used to predict whether a patient is likely to respond to a particular therapeutic therapy.

### SUMMARY OF THE INVENTION

The present invention is based on the finding that the level of expression of the enzyme H3K4 methyltransferase MLL1 in cancer cells can be used to select individuals having cancer who are likely to respond to treatment with a therapeutically effective amount of at least one compound targeting, decreasing or inhibiting the intrinsic ATPase activity of Hsp90 and/or degrading, targeting, decreasing or inhibiting the Hsp90 client proteins via the ubiquitin proteosome pathway. Such compounds will be referred to as “Heat shock protein 90 inhibitors” or “Hsp90 inhibitors. Examples of Hsp90 inhibitors suitable for use in the present invention include, but are not limited to, the geldanamycin derivative, Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990); or pharmaceutically acceptable salts thereof.

Specifically, it was found that reduced levels of MLL1 in a sample from an individual having cancer, can be used to select whether that individual will respond to treatment with HSP90 inhibitor compound 5-(2,4-Dihydroxy-5-isopropyl-phenyl)-4-(4-morpholin-4-ylmethyl-phenyl)-isoxazole-3-carboxylic acid ethylamide (AUY922), or a pharmaceutically acceptable salt thereof. The determining step can be performed by directly assaying a biological sample from the individual for the subject matter (e.g., mRNA, cDNA, protein, etc.) of interest.

In one aspect, the invention includes a method of selectively treating a subject having cancer, including selectively administering a therapeutically effective amount of (5-(2,4-Dihydroxy-5-isopropyl-phenyl)-4-(4-morpholin-4-ylmethyl-phenyl)-isoxazole-3-carboxylic acid ethylamide (AUY922), or a pharmaceutically acceptable salt thereof, to the subject on the basis of the subject having reduced levels of MLL1.

In another aspect, the invention includes a method of selectively treating a subject having cancer, including:
a) assaying a biological sample from the subject for the level of MLL1; and
b) selectively administering a therapeutically effective amount of (5-(2,4-Dihydroxy-5-isopropyl-phenyl)-4-(4-morpholin-4-ylmethyl-phenyl)-isoxazole-3-carboxylic acid ethylamide (AUY922), or a pharmaceutically acceptable salt thereof, to the subject on the basis that the sample has reduced levels of MLL1.

In yet another aspect, the invention includes a method of selectively treating a subject having cancer, including:
a) selectively administering a therapeutically effective amount of (5-(2,4-Dihydroxy-5-isopropyl-phenyl)-4-(4-morpholin-4-ylmethyl-phenyl)-isoxazole-3-carboxylic acid ethylamide (AUY922), or a pharmaceutically acceptable salt thereof, to the subject on the basis that the sample has a reduced levels of MLL1.

In yet another aspect, the invention includes a method of selectively treating a subject having cancer, including:
a) assaying a biological sample from the subject for the levels of MLL1;
b) thereafter selecting the subject for treatment with (5-(2,4-Dihydroxy-5-isopropyl-phenyl)-4-(4-morpholin-4-ylmethyl-phenyl)-isoxazole-3-carboxylic acid ethylamide (AUY922), or a pharmaceutically acceptable salt thereof, on the basis that the subject has reduced levels of MLL1; and
c) thereafter administering (5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof to the subject on the basis that the subject has reduced levels of MLL1.

In another aspect, the invention includes a method of selectively treating a subject having cancer, including:
a) determining for the levels of MLL1 in a biological sample from the subject, wherein the presence of reduced levels of MLL1 indicates that there is an increased likelihood that the subject will respond to treatment with the HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof; and
b) thereafter selecting the subject for treatment with the HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922) on the basis that the sample from the subject has reduced levels of MLL1.

In another aspect, the invention includes a method of selecting a subject for treatment having cancer, including determining for the levels of MLL1 in a biological sample from the subject, wherein the presence of reduced levels of MLL1 indicates that there is an increased likelihood that the subject will respond to treatment the HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof.

In another aspect, the invention includes a method of selecting a subject for treatment having cancer, including assaying a nucleic acid sample obtained from the subject having cancer for the levels of MLL1, wherein the presence of reduced levels of MLL1 indicates that there is an increased likelihood that the subject will respond to treatment with the HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof.

In yet another aspect, the invention includes a method of genotyping an individual including detecting a genetic variant that results in an amino acid variant at position 859 of the encoded catalytic p110α subunit of PI3K, wherein a lack of variant at, position 859 indicates that (5-(2,4-Dihydroxy-5-isopropyl-phenyl)-4-(4-morpholin-4-ylmethyl-phenyl)-isoxazole-3-carboxylic acid ethylamide (AUY922) should be administered to the individual.

In yet another aspect, the invention includes a method of genotyping an individual including detecting for the absence or presence of CAA at position 2575-2577 in the catalytic p110α subunit of PI3K gene obtained from said individual, wherein the Presence of CAA indicates that the individual has an increased likelyhood of responding to (5-(2,4-Dihydroxy-5-isopropyl-phenyl)-4-(4-morpholin-4-ylmethyl-phenyl)-isoxazole-3-carboxylic acid ethylamide (AUY922).

In another aspect, the invention includes an HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922), or a pharmaceutically acceptable salt thereof, for use in treating cancer, characterized in that a therapeutically effective amount of said compound or its pharmaceutically acceptable salt is administered to an individual on the basis of the individual having reduced MLL1 levels compared to a control at one or more of the following positions;
(a) 146982000-146984500 on chromosome X of an FMR1 genomic locus;
(b) 146991500-146993600 on chromosome X of an FMR1 genomic locus;
(c) 146994300-147005500 on chromosome X of an FMR1 genomic locus; or
(d) 147023800-147027400 on chromosome X of an FMR1 genomic locus.

In yet another aspect, the invention includes an HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922), or a pharmaceutically acceptable salt thereof, for use in treating cancer, characterized in that a therapeutically effective amount of said compound or its pharmaceutically acceptable salt is administered to an individual on the basis of a sample from the individual having been determined to have reduced levels of MLL1 compared to a control at one or more of the following positions:
(a) 146982000-146984500 on chromosome X of an FMR1 genomic locus;
(b) 146991500-146993600 on chromosome X of an FMR1 genomic locus;
(c) 146994300-147005500 on chromosome X of an FMR1 genomic locus; or
(d) 147023800-147027400 on chromosome X of an FMR1 genomic locus.

Also in the methods of the invention as described herein the cancer can be any cancer including glioblastoma; melanoma; ovarian cancer; breast cancer; lung cancer; non-small-cell lung cancer (NSCLC); endometrial cancer, prostate cancer; colon cancer; and myeloma. Typically, the sample is a tumor sample and can be a fresh frozen sample or a parrafin embedded tissue sample.

In the methods of the invention as described herein methods of detecting gluts min e or a variant amino acid can be preformed by any method known in the art such immunoassays, immunohistochemistry, ELISA, flow cytometry, Western blot, HPLC, and mass spectrometry. In addition, in the methods of the invention as described herein methods for detecting a mutation in a nucleic acid molecule encoding the catalytic p110α subunit of the PI3K include polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, or capillary electrophoresis.

The invention further includes a method for producing a transmittable form of information for predicting the responsiveness of a patient having cancer to treatment with (5-(2,4-Dihydroxy-5-isopropyl-phenyl)-4-(4-morpholin-4-ylmethyl-phenyl)-isoxazole-3-carboxylic acid ethylamide (AUY922), comprising:
a) determining whether a subject has an increased likelihood that the patient will respond to treatment with (5-(2,4-Dihydroxy-5-isopropyl-phenyl)-4-(4-morpholin-4-ylmethyl-phenyl)-isoxazole-3-carboxylic acid ethylamide (AUY922), wherein the subject has an increased likelihood based on having reduced levels of MLL1, and
b) recording the result of the determining step on a tangible or intangible media form for use in transmission.

In yet another aspect, the invention includes a kit for determining if a tumor is responsive for treatment with the HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof comprising providing one or more probes or primers for detecting the presence of a mutation at the PI3K gene locus (nucleic acid 2575-2577 of SEQ ID NO:2) and instructions for use.

In another aspect, the invention includes a kit for predicting whether a subject with cancer would benefit from treatment with the HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof, the kit comprising:
a) a plurality of agents for determining for the presence of a mutation that encodes a variant at position 859 of the catalytic p110α subunit of PI3K; and
b) instructions for use.

In the methods of the invention as described herein, the HSP90 inhibitor is any known compound targeting, decreasing or inhibiting the intrinsic ATPase activity of Hsp90 and/or degrading, targeting, decreasing or inhibiting the Hsp90 client proteins via the ubiquitin proteosome pathway. Such compounds will be referred to as “Heat shock protein 90 inhibitors” or “Hsp90 inhibitors. Examples of Hsp90 inhibitors suitable for use in the present invention include, but are not limited to, the geldanamycin derivative, Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990). In particular the compound can be 5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof; shown also below as formula (A)

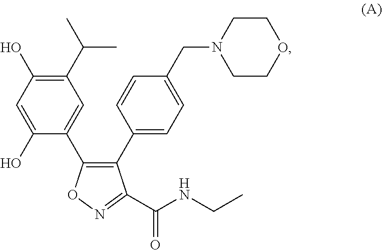 [SMILES: CCNC(=O)c1noc(-c2cc(C(C)C)c(O)cc2O)c1-c1ccc(CN2CCOCC2)cc1]

or a pharmaceutically acceptable salt thereof.

In another aspect, the invention includes a kit for determining if a tumor is responsive for treatment with the HSP90 inhibitor compound (5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof comprising providing one or more probes or primers for detecting the presence or absence of a mutation that encodes a variant in the catalytic p110α subunit of the PI3K gene at position 859.

### DESCRIPTION OF THE FIGURES

FIG. 1: Identification of MLL1 as a novel co-regulator of HSF1 in response to HSP90 inhibition by siRNA screening

A. The schematic of siRNA screening experiment design. B. Scatter plots of each siRNA hits read counts from samples treated with 100 nM AUY922 or control dimethyl sulfoxide (DMSO) samples. Each dot in the plot represents one individual siRNA hit. The cut off line was based on more than 70% luciferase activity reduction and less than 30% cell viability reduction after HSP90 inhibitor and siRNA treatment. C. A375 cell transfected with HSP70 promoter or HSP70(mHSF1) promoter-driven luciferase reporter were treated with siRNA for 3 days, then following by AUY922 treatment for one hour and then harvested to perform luciferase assay. D. A375 cell transfected with HSP70 promoter-driven luciferase reporter were treated with siRNA for 3 days, then cells were heat shock (42° C. for 30 min) and returned to 37° C. for one hour and then harvested to perform luciferase assay. E. MLL1 interacts with HSF1 in HSF1 overexpressed A375 cells. A375 cells transduced with HSF1-HA over-expression inducible lentivirus were treated with Doxycyclinefor 3 days, and then following treated or untreated with AUY922 for 6 hr. Nuclear cell extracts from A375 cells were immunoprecipitated with MLL1-C antibody or anti-HA coupled beads. Precipitated immunocomplexes were fractionated by PAGE and western blottingting with antibodies against HSF1 or MLL1-C. F. The component of MLL1 complex interacts with HSF1. G. MLL1 interacts with HSF1 in A375 cells. A375 cells were treated or untreated with AUY922 for 6 hr. Nuclear cell extracts from A375 cells were immunoprecipitated with MLL1-C or HSF1 antibody. Precipitated immunocomplexes were fractionated by PAGE and western blottingting with antibodies against HSF1 or MLL1-C.

FIG. 2 MLL1 regulates HSF1-dependent transcriptional activity and binds to HSF1 target gene promoter under HSP90 inhibition

A. Heat map showing that genes were up-regulated by AUY922, but the upregulation was impaired by MLL1 knockdown. shMLL1 transduced A375 cells were treated with or without Doxycycline for 3 days and were further treated with AUY922 100 nM for 3 h. Total RNA were collected and microarray was performed. B. Real-time PCR analysis of the expression of HSP70 and BAG3 in cells under HSP90 inhibitor treatment with or without MLL1 knockdown. ChIP with MLL1 antibody in cells treated with AUY922. shMLL1 transduced A375 cells were treated with or without Doxycycline for 3 days and were further treated with AUY922 100 nM for 1 h. Chromatin was immunoprecipitated with anti-MLL1 antibody and amplified by quantitative real-time PCR using primers around HSE element of HSP70(C) or BAG3 (D) gene promoter and MLL1 binding site of MESI1 (E) promoter. Chromatin was also immunoprecipitated with anti-H3K4me2 (F), anti-H3K4me3 (F) and anti-H4K16ac (G) antibody and amplified by quantitative real-time PCR using primers around HSE element of BAG3 gene promoter.

FIG. 3 MLL1 deficiency impairs HSF1-mediated cell response to HSP90 inhibition

A. Heat map showing that genes were up-regulated by AUY922, but the upregulation was impaired by MLL1 knockout. MLL1^{+/+} or MLL1^{-/-} MEFs were treated with or untreated with AUY922 100 nM for 3 h. Total RNA were collected and microarray was performed. B. Real-time PCR analysis of the expression of HSP70 and BAG3 in cells under HSP90 inhibitor treatment between MLL1^{+/+}and MLL1^{-/-} MEFs. MLL1^{+/+} or MLL1^{-/-} MEFs were treated with or untreated with AUY922 100 nM for 3 h. Total RNA were collected and real-time PCR were performed. C. Western blotting analysis of MLL1^{+/+} or MLL1^{-/-} MEFs with different doses of AUY922. MLL1″ or MLL1^{-/-} MEFs were treated with or without different doses of AUY922. Total protein was collected and western blottingting was performed by indicated antibodies. D. Model of the MLL1 regulated transcriptional activity as a cofactor of HSF1 during cell response to HSP90 inhibition. MLL1 and its complex bind to HSF1 and help with the transcription under HSP90 inhibition.

FIG. 4 MLL1 knockdown or knockout sensitizes cells to HSP90 inhibition

A. Cell colony formation assay of MLL1 knockdown with AUY922 treatment in A375 cells. 5000 shNTC or shMLL1 A375 cells were seeded in six wells plate and were treated or untreated with Doxycycline for 5 days, then followed by compound treatment for 6 days. B. Cell colony formation assay of MLL1 knockdown with AUY922 treatment in A2058 cells. 5000 shNTC or shMLL1 A2058 cells were seeded in six wells plate and were treated or untreated with Doxycycline for 5 days, then followed by compound treatment for 6 days. C. Western blotting analysis of tumor samples. Tumor samples were collected at the end of studies and western blotting analysis of MLL1 and GAPDH were performed. D. Real-time PCR analysis of tumor samples. Tumor samples were collected at the end of studies and total mRNA was collected and Real-time PCR was performed. E. The combinational effect of MLL1 knockdown and HSP90 inhibitor in A375 xenograft mouse model. Tumor growth rate of A375 cells expressing inducible control shRNA or shRNA against MLL1 under Doxycycline and/or HSP990 were compared at different time points. F. Cell cycle analysis of A375 cells with MLL1 knockdown and AUY922 treatment. shMLL1 transduced A375 cells were treated with or without Doxycycline for 3 days and were further treated with AUY922 100 nM for 48 h. The percentage of S+G2M cells were determined by PI staining. G. Cell apoptosis analysis of A375 cells with MLL1 knockdown and AUY922 treatment. shMLL1 transduced A375 cells were treated with or without Doxycycline for 3 days and were further treated with AUY922 100 nM for 48 h. The apoptotic cells represented by 7AAD+AnnexinV+ were determined by FACS. H. Microscopic analysis of MLL1^{+/+} and MLL1^{-/-} MEFs treated or untreated with AUY922 (25 nM or 100 nM) for 48 h. I. Dose response of AUY922 in MLL1^{+/+} or MLL1^{-/-} MEFs. MLL1^{+/+} or MLL1^{-/-} MEFs were treated with DMSO or serial dilutions of AUY922 for 24 h and 48 h. Relative cell growth was measured by CTG. J. Cell apoptosis analysis of MLL1^{+/+} or MLL1^{-/-} MEFs with AUY922 treatment. MLL1″ or MLL1^{-/-} MEFs were treated or untreated with AUY922 100 nM for 48 h. The apoptotic cells represented by 7AAD+AnnexinV+ were determined by FACS.

FIG. 5 MLL1 low expression human leukemia cells are sensitive to HSP90 inhibition

A. Real-time PCR analysis of different human leukemia cell lines under HSP90 inhibitor treatment. Human leukemia cells were cultured and treated or untreated with AUY922 for 48 h. Then, total mRNA was collected and Real-time PCR was performed. B. Cell apoptosis analysis of MLL1 low expression or MLL1 high expression human leukemia cells with AUY922 treatment. Human leukemia cells were treated or untreated with AUY922 100 nM for 48 h. The apoptotic cells represented by 7AAD+AnnexinV+ were determined by FACS. C. The effect of HSP90 inhibitor in SEM and MOLM13 xenograft mouse model. Tumor growth rate of SEM and MOLM13 under HSP990 treatment were compared at different time points. D. Heat map showing that genes were up-regulated by AUY922 in SEM cells but in MOLM13 cells. SEM and MOLM13 cells were treated or untreated with AUY922 100 nM for 3 h. Total RNA were collected and microarray was performed. E. Venn diagram showed that HSF1 activation pathway and other four signal pathways were shared by three gene profile datasets including human leukemia cells, melanoma and MEFs.

FIG. 6 Human primary B acute lymphoblastic leukemia cells with low MLL1 expression are sensitive to HSP90 inhibition

A. The percentage of human leukemia cells in bone marrow of recipient mice transplanted with human primary leukemia cells. The human cells represented by human CD45+ were determined by FACS. B. Real-time PCR analysis of MLL1 expression among different human primary leukemia cell. C. Dose response of AUY922 in human primary BALL cells. Human primary BALL cells were treated with DMSO or serial dilutions of AUY922 for 48 h. Relative cell growth was measured by CellTiter-Glo. D. JURKAT, SEM, RS(4,11) and MOLM13 were treated for 72 h with different doses of AUY922 and/or NVP-JAE067, inhibition of cell viability was measured using the CellTiter-Glo assay. E. Chalice software was used to calculate excess inhibition over Loewe additivity for each AUY922 and NVP-JAE067 dose combination.

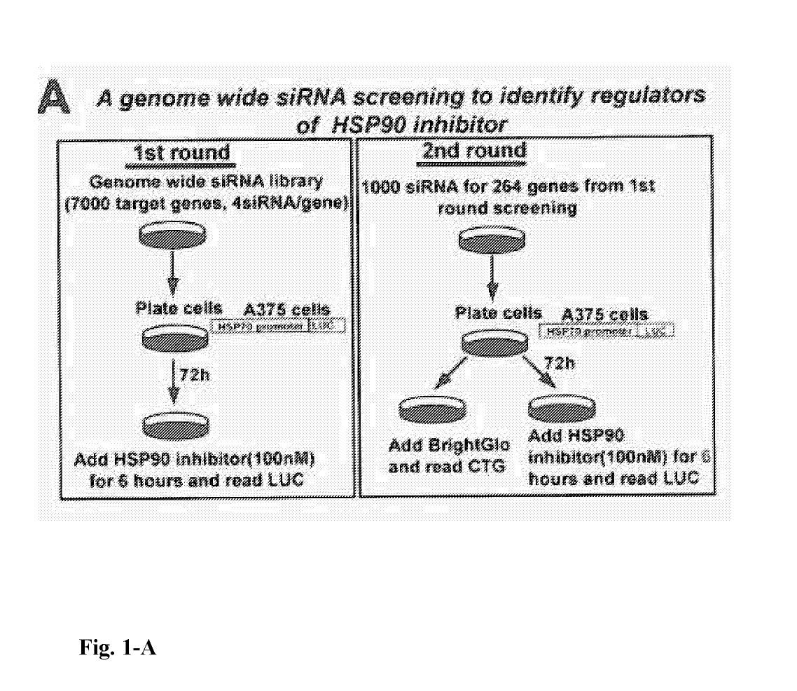

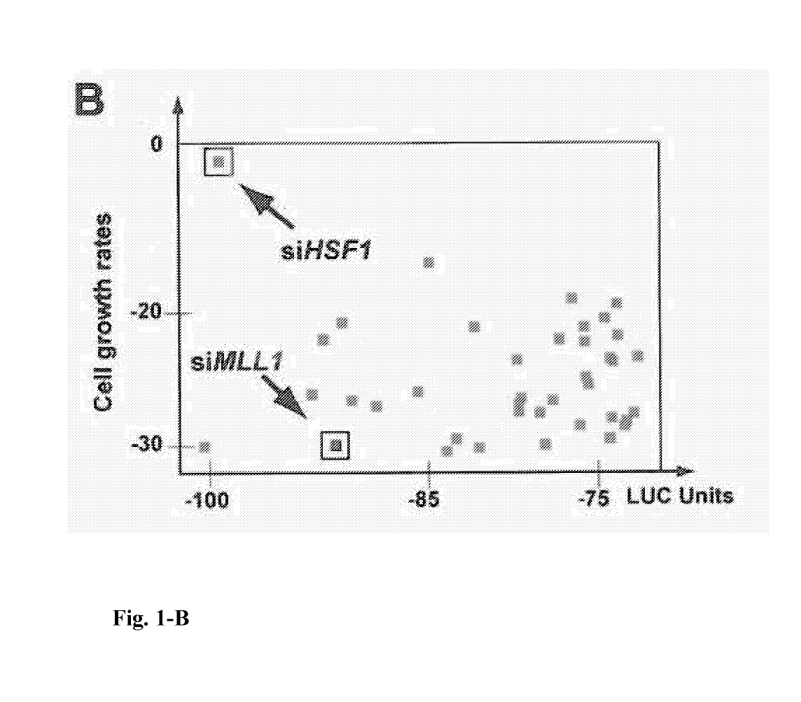

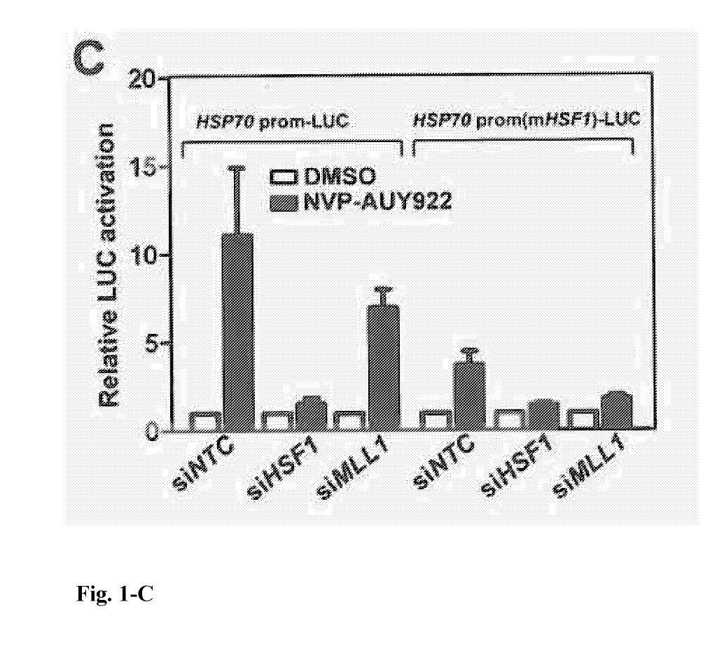

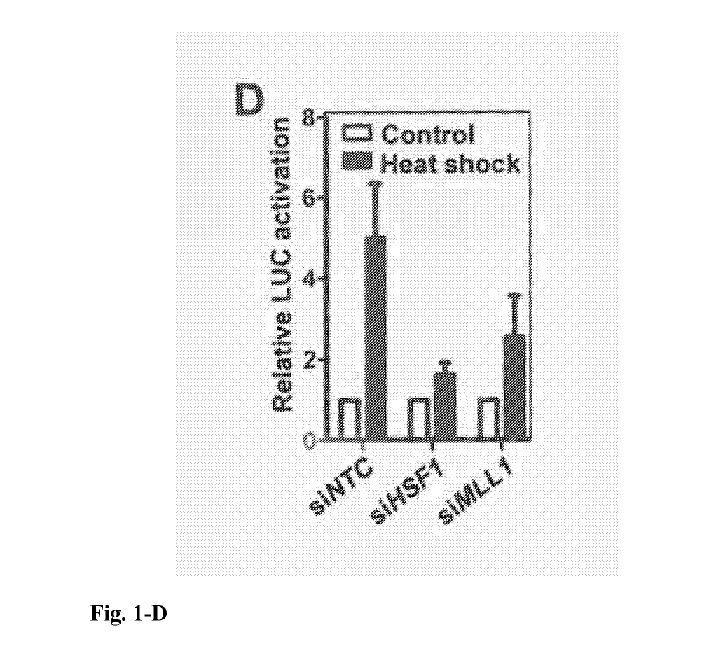

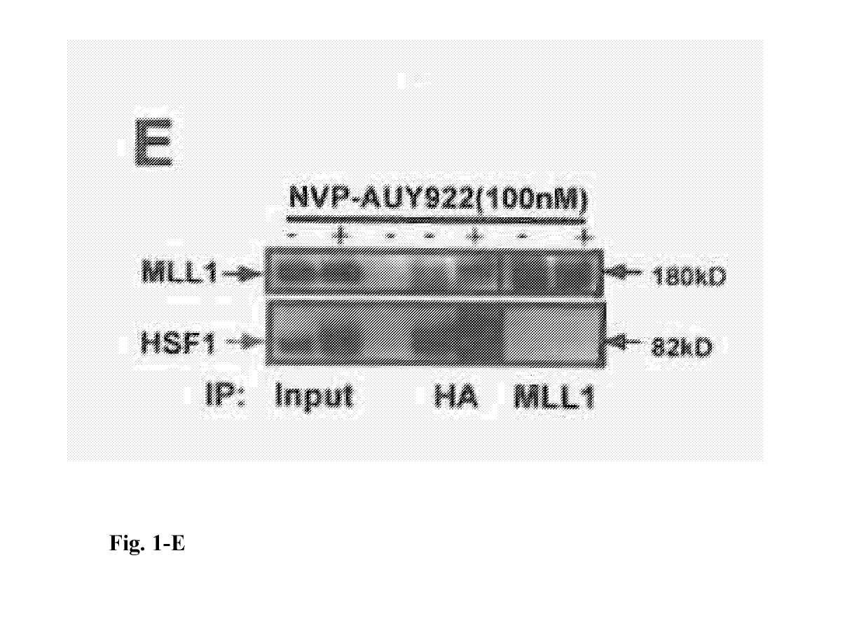

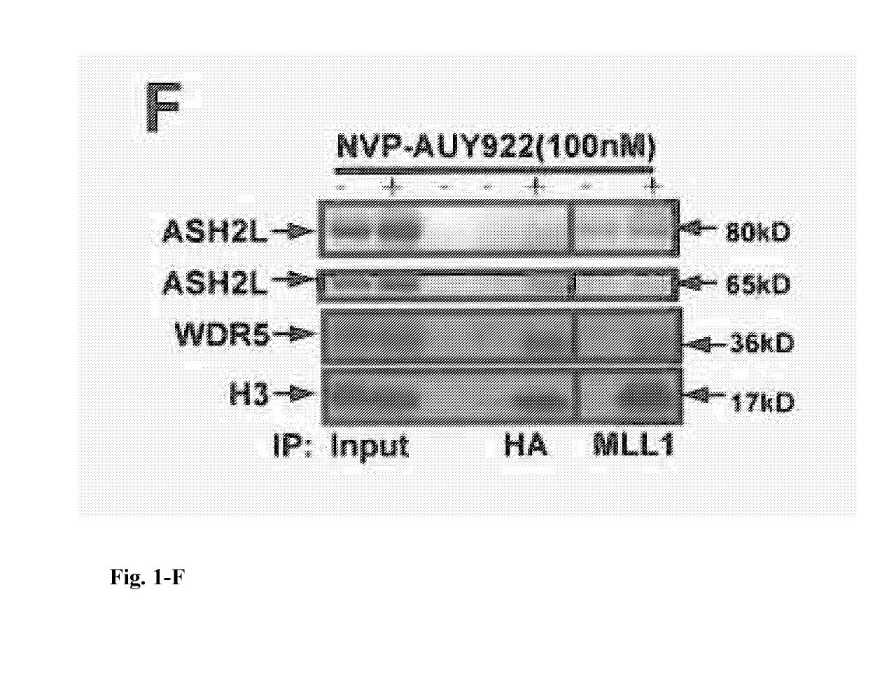

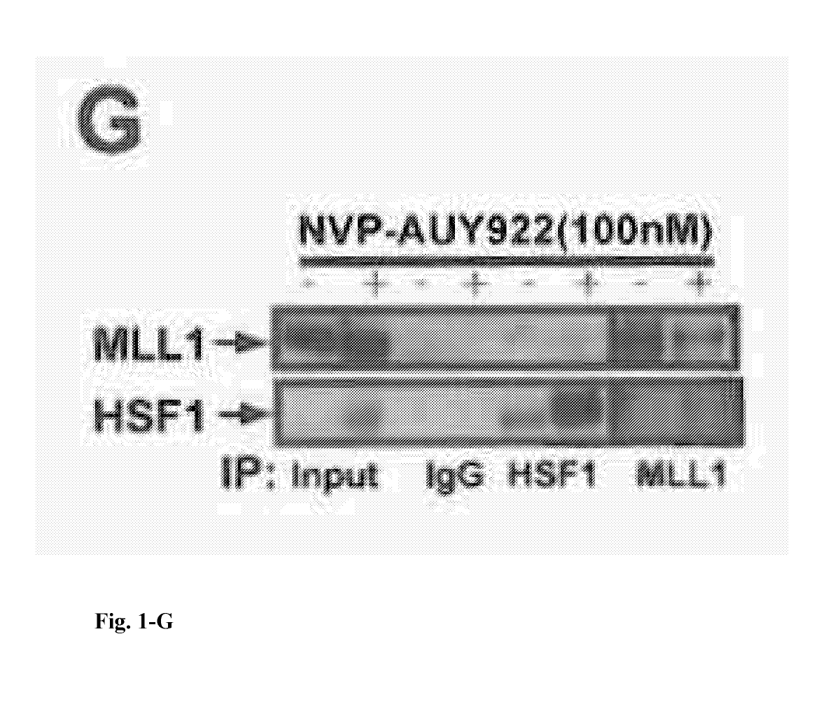

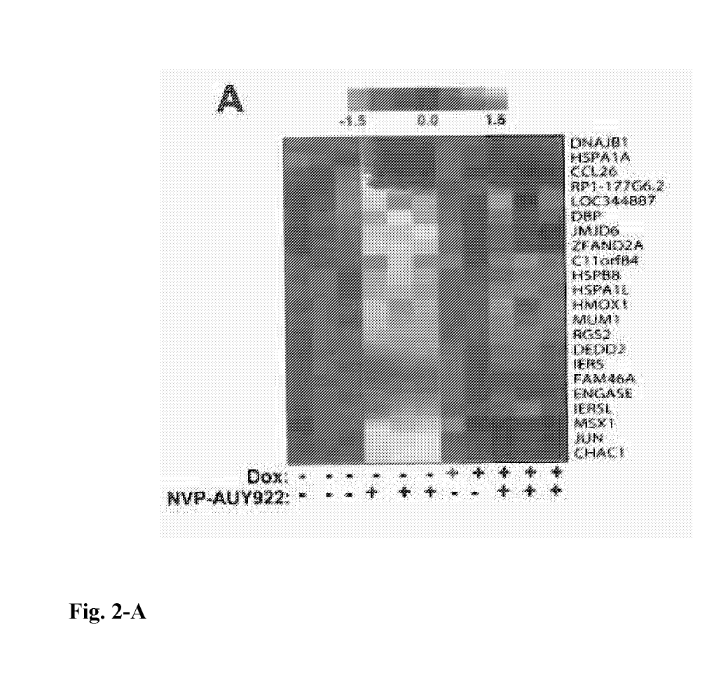

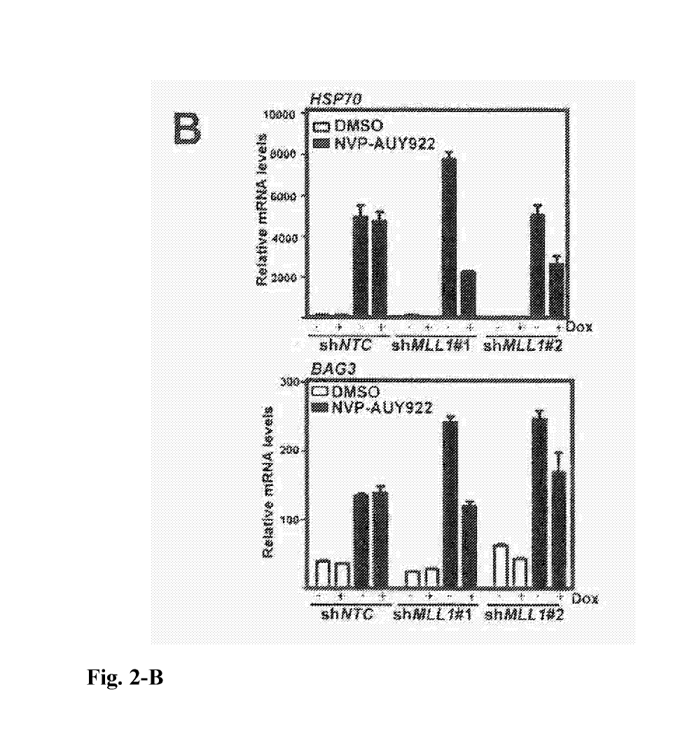

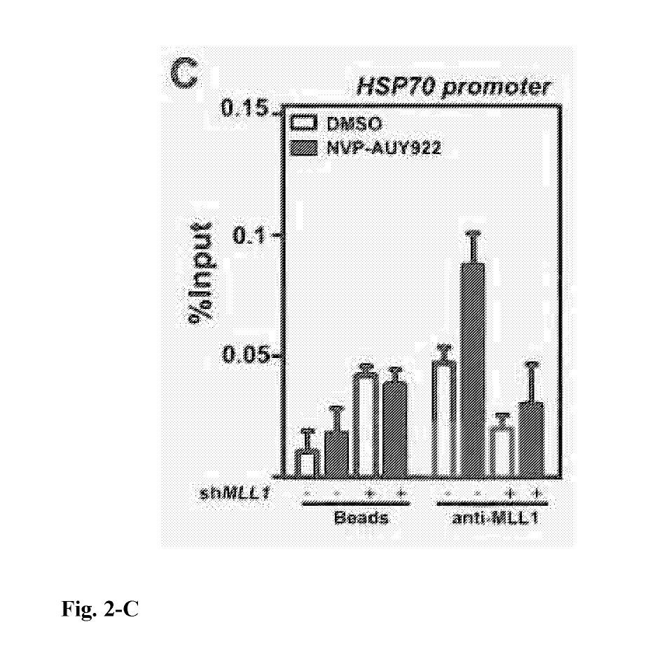

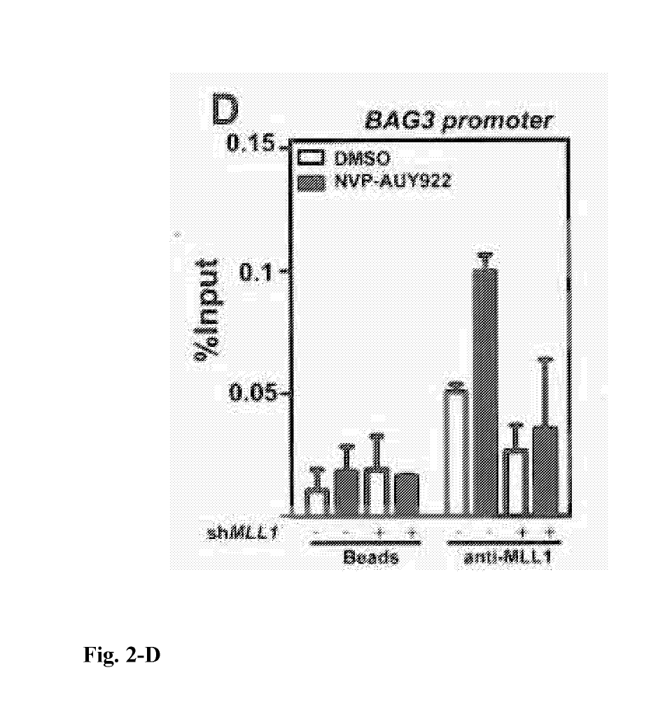

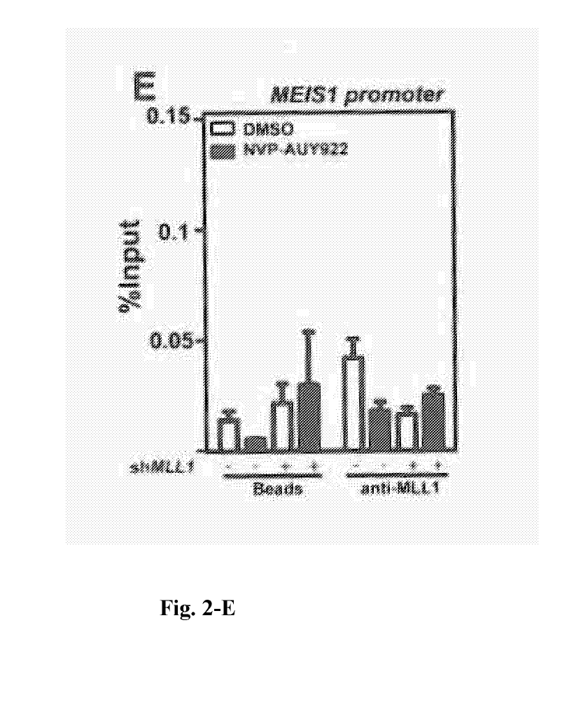

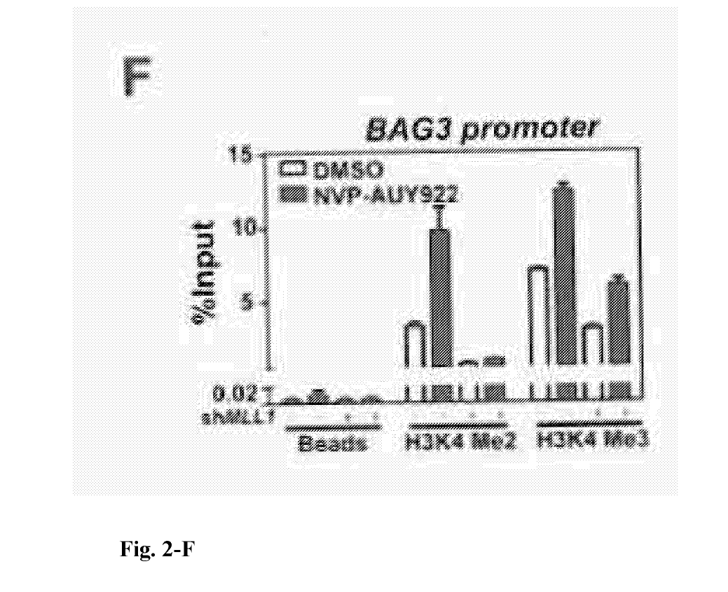

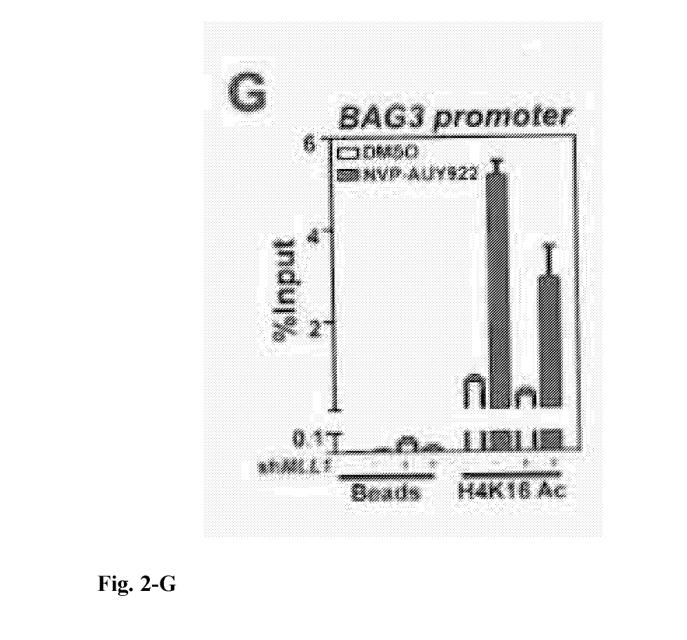

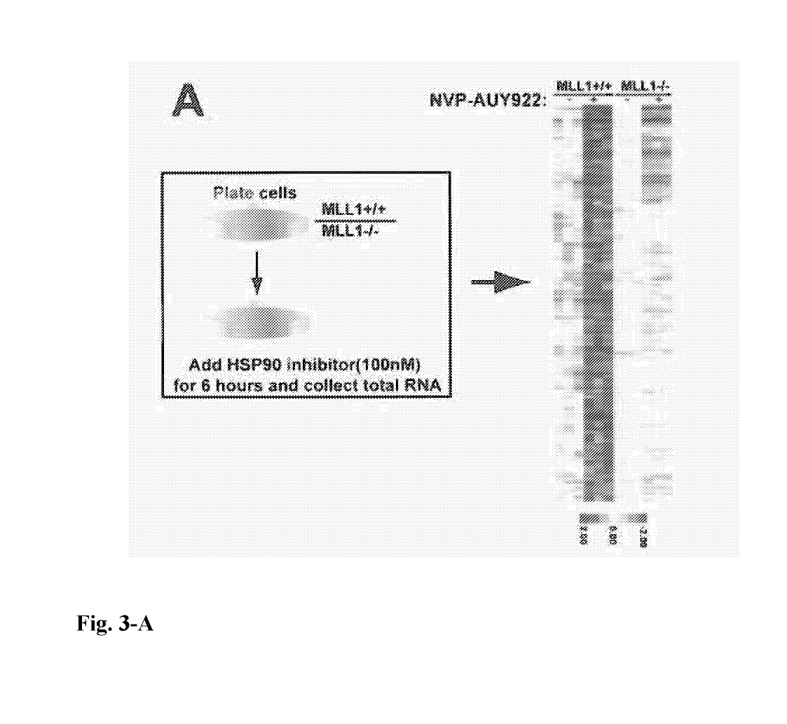

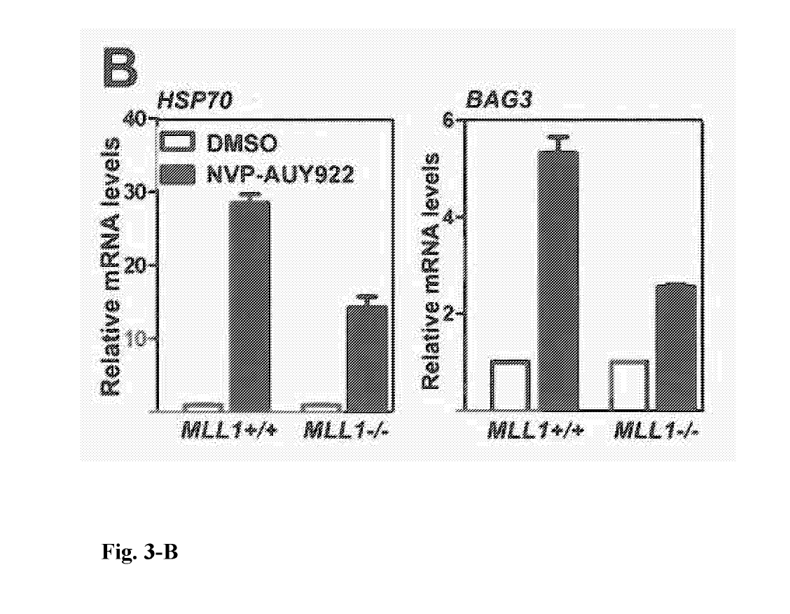

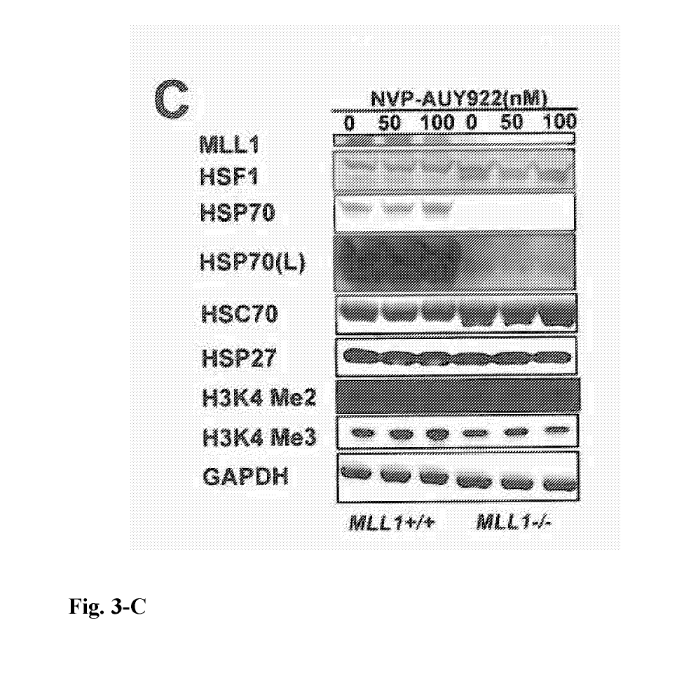

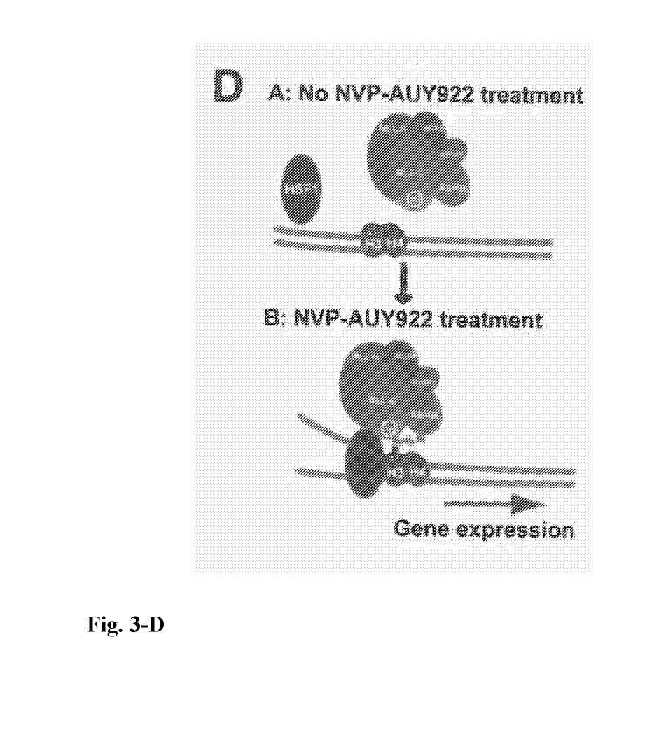

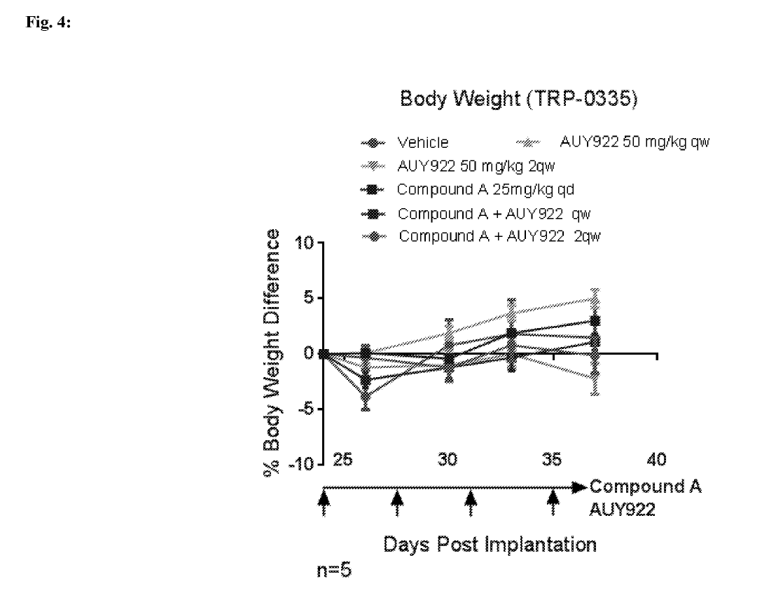

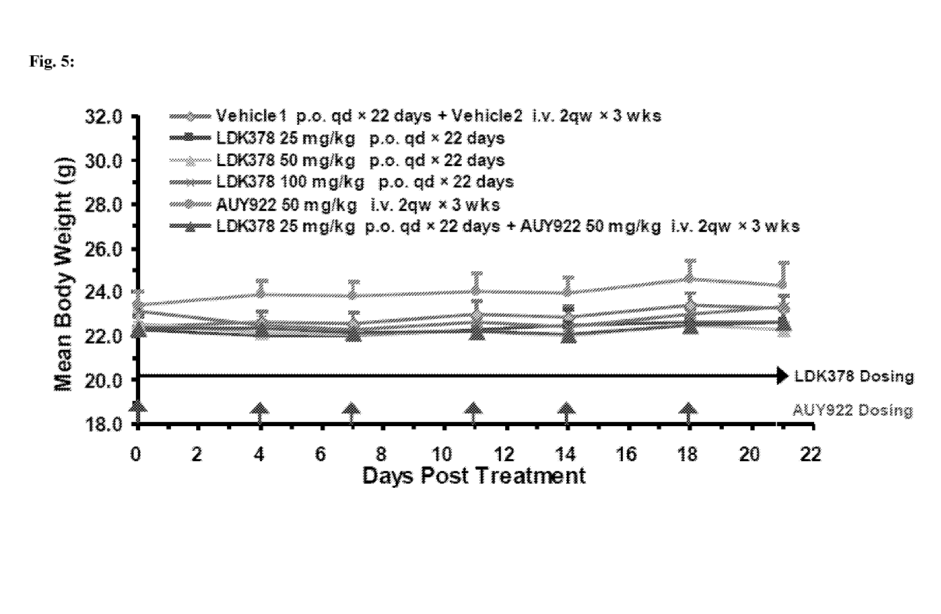

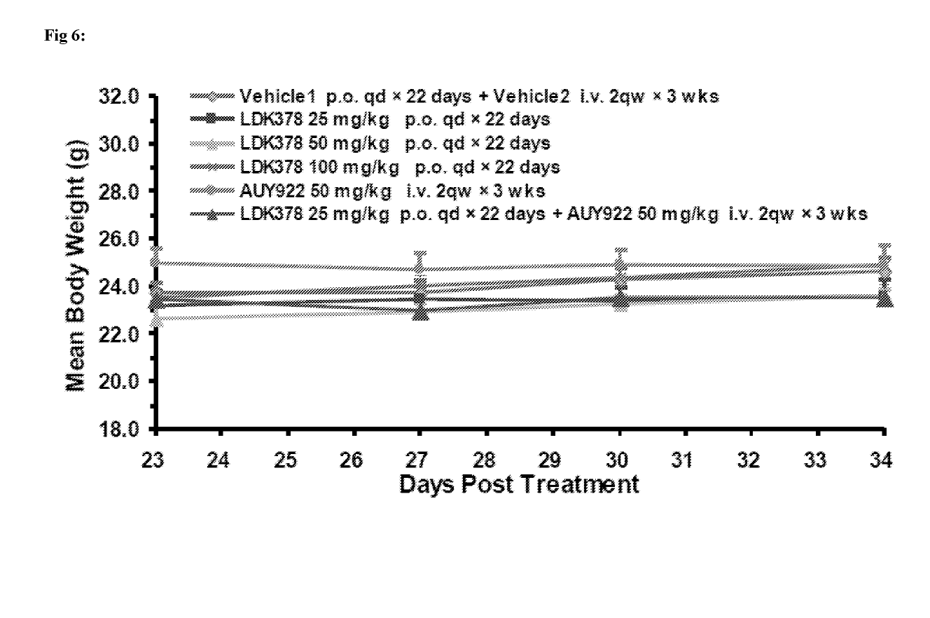

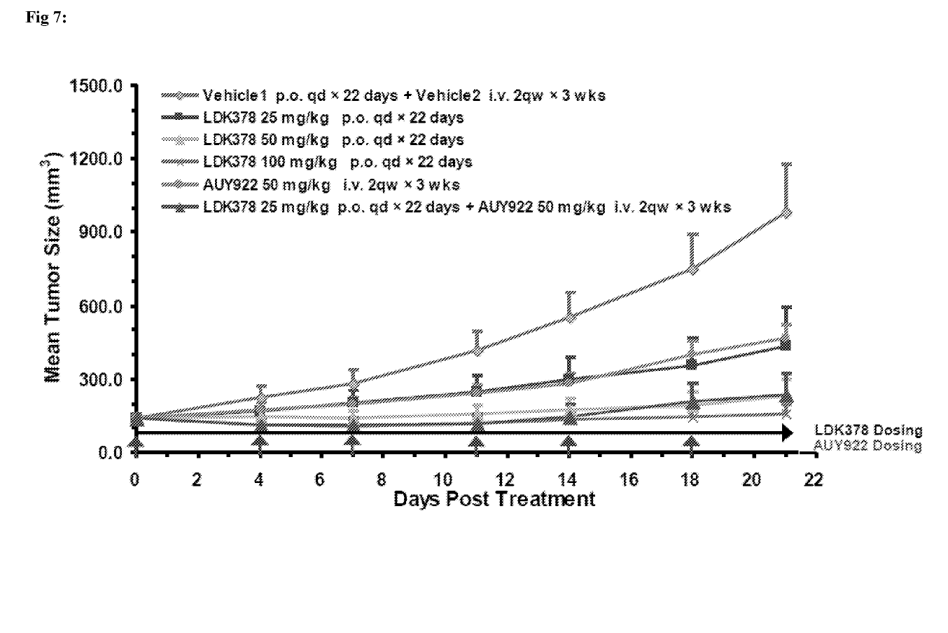

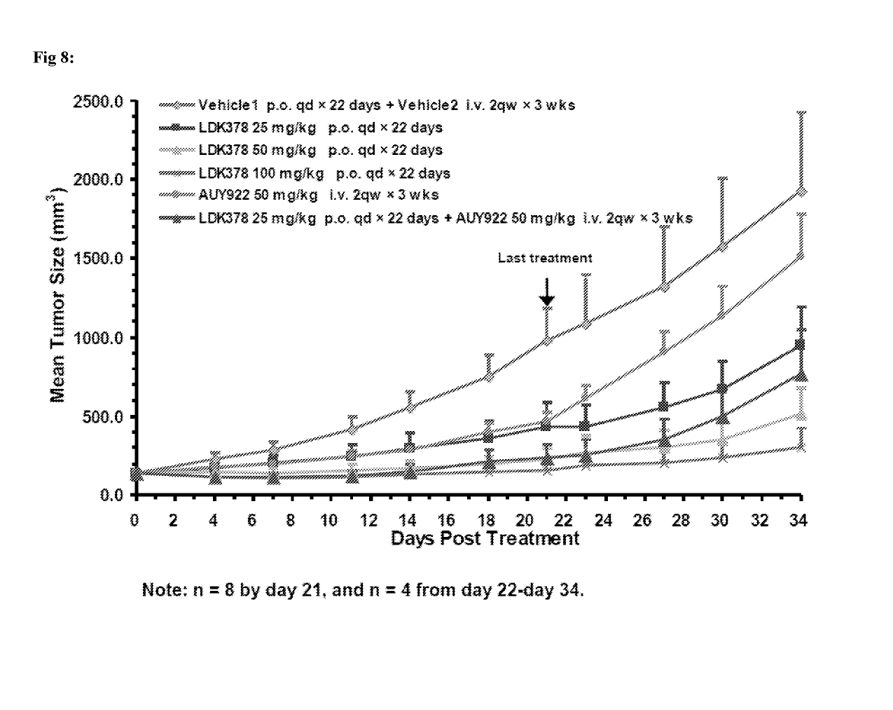

Supplementary FIG. 1: Real-time PCR and Western blotting analysis of MLL1 expression in A375 cells with inducible MLL1 knockdown

shNTC or shMLL1 transduced stable cell lines were treated with Doxycycline for 3 days and cell pellets were collected and Real-time PCR and western blotting were performed.

Supplementary FIG. 2: MLL1 knockdown didn't affect HSF1 expression at both mRNA level and protein level

Supplementary FIG. 3: ChIP with HSF1 antibody in cells treated with AUY922

shHSF1 transduced A375 cells were treated with or without Doxycycline for 3 days and were further treated with AUY922 100 nM for 1 h. Chromatin was immunoprecipitated with anti-MLL1 antibody and amplified by quantitative real-time PCR using primers around HSE element of HSP70 (A) or BAG3 (B) gene promoter and MLL1 binding site of MESI1 (C) promoter.

Supplementary FIG. 4: Cell colony formation assay of MLL1 knockdown with AUY922 treatment in HCT116 cells

5000 shNTC or shMLL1 HCT116 cells were seeded in six wells plate and were treated or untreated with Doxycycline for 5 days, then followed by compound treatment for 6 days.

Supplementary FIG. 5: Cell colony formation assay of HSF1 knockdown or MLL1 knockdown with NVP-LGX818 treatment in A375 cells 5000 shNTC, shHSF1 or shMLL1 A375 cells were seeded in six wells plate and were treated or untreated with Doxycycline for 5 days, then followed by compound treatment for 6 days.

Supplementary FIG. 6: Western blotting analysis of A375 cells expressing the inducible shMLL1 treated with different doses of AUY922

shNTC or shMLL1 transduced A375 cells were treated with or without Doxycycline for 3 days and were further treated with different doses of AUY922 for 48 h.

Supplementary FIG. 7: Real-time PCR analysis of MLL1 expression among human leukemia cells

### DETAILED DESCRIPTION OF THE INVENTION

“Treatment” includes prophylactic (preventive) and therapeutic treatment as well as the delay of progression of a disease or disorder. The term “prophylactic” means the prevention of the onset or recurrence of diseases involving proliferative diseases. The term “delay of progression” as used herein means administration of the combination to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

“Subject” is intended to include animals. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a brain tumor disease. Particularly preferred, the subject is human.

“Pharmaceutical preparation” or “pharmaceutical composition” refer to a mixture or solution containing at least one therapeutic compound to be administered to a mammal, e.g., a human in order to prevent, treat or control a particular disease or condition affecting the mammal.

“Co-administer”, “co-administration” or “combined administration” or the like are meant to encompass administration of the selected therapeutic agents to a single patient, and are intended to include treatment regimens in which the agents are not necessarily administered by the same route of administration or at the same time.

“Pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

“Therapeutically effective” preferably relates to an amount that is therapeutically or in a broader sense also prophylactically effective against the progression of a proliferative disease.

“Single pharmaceutical composition” refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

“Dose range” refers to an upper and a lower limit of an acceptable variation of the amount of agent specified. Typically, a dose of the agent in any amount within the specified range can be administered to patients undergoing treatment.

The terms “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

Here, we established a derivative of human melanoma cells with integrated HSP70 promoter-driven luciferase reporter and performed a genome wide druggable siRNA screen to look for the co-modulators of HSF1. We identify that the H3K4 methyltransferase MLL1 works as a co-factor of HSF1 in cell response to HSP90 inhibition. MLL1 interacts with HSF1, binds to the promoter of HSF1-target genes and regulates HSF1-dependent transcriptional activation under HSP90 inhibition. A striking combinational effect was observed when MLL1 knockdown or knockout in combination with HSP90 inhibition in various cell lines and tumor mouse models. Our data indicate that MLL1 is a cofactor of HSF1 and establish a critical role for MLL1 in cell response to HSP90 inhibition.

Chromosomal translocations that disrupt the Mixed Lineage Leukemia protein-1 gene (MLL1, ALL1, HRX, Htrx)) are associated with a unique subset of acute lymphoblastic or myelogenous leukemias [1-4]. The product of MLL1 gene is a large protein that functions as a transcriptional co-activator required for the maintenance of Hox gene expression patterns during hematopoiesis and development [5-8]. The transcriptional co-activator activity of MLL1 is mediated in part by its histone H3 lysine 4 (H3K4) methyltransferase activity [6], an epigenetic mark correlated with transcriptionally active forms of chromatin [9, 10]. MLL1 complexes catalyze mono-, di- and trimethylation of H3K4, the regulation of which can have distinct functional consequences.

The present invention comprises At least one compound targeting, decreasing or inhibiting the intrinsic ATPase activity of Hsp90 and/or degrading, targeting, decreasing or inhibiting the Hsp90 client proteins via the ubiquitin proteosome pathway. Such compounds will be referred to as “Heat shock protein 90 inhibitors” or “Hsp90 inhibitors. Examples of Hsp90 inhibitors suitable for use in the present invention include, but are not limited to, the geldanamycin derivative, Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990).

### Results:

Identification of MLL1 as a Co-Regulator of HSF1 in Response to HSP90 Inhibition by siRNA Screening

To identify the novel co-regulator of HSF1 in response to HSP90 inhibition, we established a derivative of A375 cells with integrated HSP70 promoter-driven luciferase reporter activated by HSP90 inhibitor treatment and performed two rounds siRNA screen (FIG. 1A). To perform a high-throughput genome-wide druggable targets siRNA screen, the full siRNA library containing 7000 genes was stamped out in 384 well plates, as well as HSF1 siRNA and negative controls. siRNA screening were performed for two rounds. Luciferase activity was used to select gene for second round screen. Top 1000 siRNAs for 264 genes from the 1^{st} round screen were selected to perform the 2^{nd} round screen. For the 2^{nd} round screen, both luciferase activity and cell viability were measured. The counter screening assays, for example, examining the endogenous HSP70 gene expression after knockdown of potential HSF1-modulators selected from above screen and examining potential HSF1-modulators genes knockdown, were also performed. The cut off line was based on more than 70% luciferase activity reduction and less than 30% cell viability reduction after HSP90 inhibitor and siRNA treatment. 35 genes were found to meet the criteria (Supplementary Table. 1) and among those genes, MLL1, MED6, MED19, MED21, and SMARCD3 are known as chromatin remodeling factors. MLL1 is a known H3K4 methyltransferase and involved in gene transcriptional activity. HSF1 knockdown didn't affect cell proliferation, but inhibited 100% luciferase activity. MLL1 knockdown inhibited less than 30% cell proliferation, but reduced more than 90% luciferase activity (FIG. 1B). To validate that MLL1 could participate in the regulation of cell response to HSP90 inhibition, we knocked down MLL1 in A375 cells with HSP70 promoter reported plasmid by using different sequence small interfering RNA (siRNA). (how is the sequence different?) This reduces the expression of the MLL1 gene by treatment with a siRNA reagent with a sequence complementary to the mRNA transcript of the MLL1 gene. The binding of this siRNA to the active MLL1 gene's transcripts causes decreased expression through degredation of the mRNA transcripts). MLL1 knockdown repressed more than 40% luciferase activity caused by HSP90 inhibition while HSF1 knockdown repressed about 90% luciferase activity (FIG. 1C). We further determined whether MLL1 regulated cell response to HSP90 inhibition through HSF1 by mutating the HSF1 binding site in HSP70 promoter. As expected, more than 70% reduction of HSP90 inhibition induced luciferase activity was observed when one HSF1 binding site in HSP70 promoter was mutated. Interestingly, MLL1 knockdown with HSF1 binding site mutation repressed more than 80% luciferase activity (FIG. 1C). Those results suggested that MLL1 participated in the regulation of cell response to HSP90 inhibition. The idea that MLL1 could regulate the heat shock response was also tested under heat shock condition. Similar with HSP90 inhibition, heat shock induced HSP70 promoter luciferase activity. HSF1 knockdown inhibits heat shock response while MLL1 knockdown reduced more than 40% heat shock induced luciferase activity (FIG. 1D). To explore whether MLL1 and its complex bind to HSF1 under HSP90 inhibition, we performed co-immunoprecipitation assay with cells transduced with control or HSF1-HA construct. Western blotting showed the presence of MLL1 with HSF1 under HSP90 inhibition. Reverse co-immunoprecipitation assays showed that HSF1 epitopes also precipitated MLL1 protein (FIG. 1E). In addition, western blotting showed the MLL1 complex components: ASH2L and WDR5 also precipitated HSF1 or MLL1 (FIG. 1F). To test for in vivo interactions between endogenous HSF1 and MLL1, nuclear protein extracts from A375 cells treated with or without HSP90 inhibitor were immunoprecipitated with HSF1 or MLL1 antibodies. Western blotting revealed the presence of MLL1 or HSF1 in the anti-HSF1 or anti-MLL1 immunoprecipitates (FIG. 1G).

### MLL1 Regulates HSF1-Dependent Transcriptional Activity and Binds to HSF1-Target Gene Promoter Under HSP90 Inhibition

We further tested whether MLL1 knockdown affects the HSF1-dependent transcriptional activity. We introduced shMLL1 into A375 cells and then exposed the cells to HSP90 inhibition. Gene profile analysis showed that 38 genes transcription activities were induced by HSP90 inhibition. The induction of transcription activities of 22 genes/38 genes were repressed by MLL1 knockdown to varying degree (FIG. 2A). A part of 22 genes belong to HSF1-regulated cell stress pathway, such as HSPA1A, HSPA1L, HSPB8, DEDD2 and DNAJB1 (FIG. 2A). To validate the gene profile results, two MLL1 inducible shRNA constructs by targeting distinct MLL1 sequence were stably introduced into A375 cancer cells and knockdown of MLL1 was confirmed (Supplementary FIG. 1). The MLL1 regulated HSP70 and BAG3 transcription activities under HSP90 inhibitor treatment was further validated by real-time PCR. MLL1 knockdown didn't affect HSF1 expression at both mRNA level and protein level (supplementary FIG. 2), but repressed the HSF1-target gene HSP70 and BAG3 mRNA levels under HSP90 inhibitor treatment (FIG. 2B).

To examine the recruitment of MLL1 to the HSF1-modulated gene promoter, we performed chromatin immunoprecipitation (ChIP) with A375 cells transduced with control or shMLL1 and treated or untreated with AUY922 for 1 h. Chromatin from those cells was sonicated to obtain fragments below 500 bp and immunoprecipitated using polyclonal against HSF1 and MLL1. Quantitative real-time PCR analysis was carried out with primer specific for the HSP70 and BAG3 encompassing the HSE element. MLL1 binding site of MESI1 was used as a control. We observed that the binding of HSF1 to HSP70 or BAG3 promoter, but not MESI1, increased about ten times at one hour of AUY922 treatment (Supplement FIG. 3). In contrast, the bindings were not detected in A375 cells with inducible HSF1 knockdown (Supplement FIG. 3). A significant of MLL1 occupancy of the HSP70 and BAG3 gene promoter is also observed at one hour of AUY922 treatment (FIGS. 2C and D). In contrast, the binding of MLL1 to MESI promoter was not detected (FIG. 2E). The MLL1 bindings were significantly reduced by MLL1 knockdown (FIGS. 2C, D and E). As MLL1 mediates the Di- and Tri-methylation of Lys-4 of histone H3 (H3K4me) and acetylation of Lys-16 of histone H4 (H4K16ac), we next examined whether H3K4me2, H3K4me3 and H4K16ac are recruited to HSF1-regulated gene promoter under HSP90 inhibition. We observed that H3K4me2 and H3K4me3 bound to BAG3 promoter and those bindings were further significantly enhanced by AUY922 treatment, while diminished by MLL1 knockdown (FIG. 2F). Similarly, H4K16ac also bound to BAG3 promoter and those bindings were further significantly enhanced by AUY922 treatment, while diminished by MLL1 knockdown (FIG. 2G). Taken together, these data suggest that MLL1 regulates HSF1-dependent transcriptional activity and binds to HSF1 target-gene promoter under HSP90 inhibition.

### MLL1 Deficiency Impairs HSF1-Mediated Cell Response to HSP90 Inhibition

To further validate the shRNA results, we next examined the MLL1^{-/-} mouse embryonic fibroblast (MEFs) response to HSP90 inhibition. Gene profile analysis showed that the transcription activities of 68 genes were induced by HSP90 inhibition and the upregulation of those genes were impaired by MLL1 deficiency to varying degree (FIG. 3A). A part of those genes also belong to HSF1-regulated cell stress pathway, such as Dnaja1, Dnajb4, DnaJ2 and Bag3. The regulation of two HSF1-target genes: Hspa1b and Bag3 by HSP90 inhibitor in MEFs was further validated by quantitative real-time PCR and loss of MLL1 led to about 50% reduction of Hspa1b or Bag3 expression under AUY922 treatment (FIG. 3B). In addition, western blotting showed that HSP90 inhibition induced the heat shock pathway in MEFs. Surprisingly, HSP70 protein level was dramatically repressed while HSC70 protein level was significantly enhanced in MLL1^{-/-} MEFs (FIG. 3C). Consistent with MLL1 deletion, the global level of H3K4me3, but not H3K4me2, was decreased in MLL1^{-/-} MEFs (FIG. 3C). These results indicate that MLL1 is a cofactor of HSF1, binds to HSF1-modulated gene promoter, mediates the Di- and Tri-methylation of H3K4me and regulates the HSF1-dependent transcriptional activity under HSP90 inhibition (FIG. 3D).

### MLL1 Knockdown or Knockout Sensitizes Cells to HSP90 Inhibition

Our previous work identified HSF1 as a key sensitizer to HSP90 inhibitor in human cancer. We next examined whether MLL1 is also a sensitizer to HSP90 inhibitor. To validate whether MLL1 was indeed a sensitizer of HSP90 inhibition, the combinational effect of MLL1 knockdown with AUY922 were tested among three cancer cell lines (A375, A2058 and HCT116). Two MLL1 inducible shRNA constructs by targeting distinct MLL1 sequence were stably introduced into different cancer cell lines. In those three cancer cell lines, induction of MLL1 shRNA as well as HSF1 shRNA (but not the NTC shRNA) led to a dramatically sensitivity to AUY922 through colony formation assays (FIG. 4A, B and Supplementary FIG. 4). In contrast, MLL1 knockdown does not have a combinational effect with BRAF inhibitor NVP-LGX818 (Supplementary FIG. 5), which suggests that MLL1 knockdown has a selective effect with HSP90 inhibitor. These findings indicate MLL1 as a valid sensitizer to HSP90 inhibition in cancer cells. To further validate MLL1 as a sensitizer of HSP90 inhibitor, we examined the combinational effect of MLL1 knockdown with HSP90 inhibitor in A375 xenograft mouse model. MLL1 shRNA alone slightly inhibit tumor growth, and knockdown was confirmed at protein level and mRNA level (FIGS. 4C and D). HSP990 alone at tolerated dosage (10 mg/kg PO, qw) inhibited tumor growth by 50% T/C (FIG. 4E). More strikingly, HSF1 knockdown & HSP990 combination led to tumor stasis (FIG. 4E). These results suggest that MLL1, a regulator of cell stress response, is also critical for limiting the efficacy of HSP90 inhibitor in human cancer cells and the combination of MLL1 knockdown, and HSP90 inhibitor is sufficient to cause the stasis of melanoma growth.

To understand the mechanism of the combination effects of MLL1 knockdown and HSP90 inhibition, we further tested whether: 1) MLL1 knockdown may facilitate the degradation of HSP90 client protein, such as BRAF; 2) MLL1 knockdown may attenuate MAPK signaling based on recent finding that HSF1 deficiency attenuates MAPK signaling in mice. We performed western blotting in cells treated with MLL1 shRNA and HSP90 inhibitor. The combination of MLL1 knockdown and HSP90 inhibitor led to a decreased level of p-ERK but not the degradation of BRAF in A375 cells (Supplementary FIG. 6). To understand how HSF1 knockdown affects the cell proliferation under HSP90 inhibitor treatment, we performed a DNA content analysis to examine the effect of MLL1 knockdown on cell cycle progression under HSP90 inhibitor treatment. Similarly with HSF1 knockdown, MLL1 knockdown didn't affect the percentage of cancer cells in cell cycle while HSP90 inhibitor caused more cancer cells into S+G2M phase (FIG. 4F). In contrast, The percentage of cancer cells in the S+G2M phase was significantly lower in MLL1 knockdown group than in the control group under HSP90 inhibitor treatment (FIG. 4F), indicating that the knockdown of MLL1 blocks cancer cells to enter the cell cycle, thereby decreasing the proliferation of cancer cells. Furthermore, we examined whether MLL1 knockdown enhances apoptosis of cancer cells under HSP90 inhibitor treatment by staining the cells with 7AAD and Annexin V. Similarly, MLL1 knockdown didn't affect the apoptosis of cancer cells while HSP90 inhibitor induced the apoptosis of cancer cells (FIG. 4G). MLL1 knockdown further enhanced the apoptotic proportion of cancer cells under HSP90 inhibitor treatment (FIG. 4G). Thus, MLL1 knockdown attenuates MAPK growth signaling, leads to cell cycles arrest and induces cell apoptosis under HSP90 inhibitor treatment. To further validate the shRNA results, we next examined whether loss of MLL1 sensitizes cells to HSP90 inhibition. In MLL1^{+/+} MEFs, AUY922 inhibits the proliferation rate of MEFs, but didn't kill those cells. In contrast, more than 90% of MLL1^{-/-} MEFs were killed by AUY922 after 48 h treatment (FIGS. 4H and I). Cell apoptosis analysis showed that more than 80% MLL1^{-/-} MEFs versus only 30% MLL1^{+/+} were induced apoptosis under AUY922 treatment (FIG. 4J). These data indicate that MLL1 is a potential target to sensitize human cancer cells to HSP90 inhibition.

### MLL1 Low Expression Human Leukemia Cells are Sensitive to HSP90 Inhibition

As knockdown or loss of MLL1 leads to an increased efficacy of HSP90 inhibitor on cell proliferation, we further tested the idea that human cancer cells with MLL1 low expression level should be more sensitive to HSP90 inhibition. In human leukemia, some fusion genes including MLL-AF4, MLL-AF9 and MLL-ENL were caused by MLL1 translocation. We first examined the MLL1 mRNA levels among nine different human leukemia cells with or without MLL1 translocations. JURKAT, 697 and REH are wild-type leukemia cells with high MLL1 expression and SEM cells carrying MLL1-AF4 also has a high MLL1 expression. PL21 cells carrying FLT3 ITD mutation, RS(4,11) cells carrying MLL1-AF4 have a relative low MLL1 expression. And NOMO1 cells carrying MLL1-AF9 and NOMO1 carrying MLL1-AF9 have lowest MLL1 expression (Supplementary FIG. 7). We next examined whether MLL1 expression associated with cell response to HSP90 inhibition. The HSP70 and BAG3 expression representing cell stress response to HSP90 inhibitor was also tested among those leukemia cells. The cell stress response to HSP90 inhibition was significantly reduced in RS(4,11) and MOLM13 cells (FIG. 5A). NOMO1 with MLL1 low expression didn't show a reduced cell stress response to HSP90 inhibition (FIG. 5A). We next tested sensitivity of each leukemia cell line to HSP90 inhibitor. IC95 of AUY922 in NOMO, MOLM13 and RS(4,11) are about 100 nM while IC95 of AUY922 in other leukemia cell lines are about 1000 nM (Table.1). Those results suggested that MLL1 expression may associate with cell sensitivity to HSP90 inhibitor, but not associate with cell response to HSP90 inhibition, which suggested that there are some MLL1 mediated mechanisms independent on HSF1-activated cell response to HSP90 inhibition. Cell apoptosis analysis showed that a higher cell apoptosis rate were induced in RS(4,11) and MOLM13 cells than in JURKAT and PL21 cells (FIG. 5B). Furthermore, we examined the effect of HSP90 inhibitor in SEM and MOLM13 xenograft mouse models. HSP990 at tolerated dosage (10 mg/kg PO, qw) inhibited SEM tumor growth by 30% T/C while inhibited MOLM13 tumor growth by 60% T/C (FIG. 5C). To test the idea that leukemia cells with low MLL1 expression may present a reduced HSF1 regulated transcriptional activity to HSP90 inhibition, we compared gene profile of SEM and MOLM13 leukemia cells response to HSP90 inhibition. Gene profile assay showed that 32 genes expression were highly induced by HSP90 inhibition in SEM, but not in MOLM13 to varying degree (FIG. 5D). All three gene profile datasets in different cells response to HSP90 inhibition including melanoma with or without MLL1 knockdown, human leukemia cells with high or low MLL1 expression and MLL1^{+/+} or MLL1^{-/-} MEFs were performed pathway analysis. HSF1 pathway activation is the most significantly shared pathway by three gene profile datasets. PRDM2 activation, BACH2 inhibition, BLVRA activation and PES1 activation are also shared by three gene profile datasets. These results indicate that MLL1 may be a potential biomarker to stratify patients in HSP90 inhibitor treatment.

Human Primary B Acute Lymphoblastic Leukemia Cells with Low MLL1 Expression are Sensitive to HSP90 Inhibition

To investigate whether MLL1 expression is different in primary human cancer cells, we examined the expression of MLL1 in human B acute lymphoblastic leukemia samples. The primary human BALL cells were transplanted into immune deficient mice and bone marrow cells were collected from recipient mice until blood tumor burden is higher than 70% by FACS analysis. Bone marrow cells were cultured and FACS analysis showed that more than 90% cells are human leukemia cells (FIG. 6A). Real-time PCR showed that MLL1 expression is three times higher in P1 patient than in P4 patient (FIG. 6B). We next evaluated the efficacy of AUY922 on those human leukemia cells. As expected, P1 leukemia cells with high MLL1 expression didn't response to AUY922 treatment while P4 leukemia cells with low MLL1 expression showed a good response to AUY922 treatment Other two human leukemia samples also showed a certain response to AUY922 (FIG. 6C). MLL1 fusion oncoproteins are known to recruit DOT1L to activate the downstream signaling pathways and leukemia cells harboring a MLL1 translocation may likely have a low wild type MLL1 expression as one wild-type MLL1 allele is lost, which suggested that those kind of leukemia cells may be sensitive to combination of HSP90 inhibitor and DOT1L inhibitor. We next tested the combination effect of AUY922 and DOT1L inhibitor NVP-JAE067 on human leukemia cells. AUY922 and NVP-JAE067 showed a significant combination effect on leukemia cells carrying MLL1 translocation including SEM, RS(4,11) and MOLM13 cells, but not on MLL1 wild type leukemia cells: JURKAT cells (FIG. 6D). Taken together, those result indicated human leukemia cells with MLL1 low expression may be more sensitive to HSP90 inhibition and the combination of HSP90 inhibitor and DOT1L inhibitor may be a good strategy for human leukemia cells harboring MLL1 translocation.

### Method and Materials:

### Cell Culture

A375, A2058, HCT116, SEM, 697, JURKAT, REH, PL21, NOMO1, RS(4,11) and MOLM13 cells were obtained from American Type culture Collection. MLL1+/+ and MLL1−/− mouse embryonic fibroblasts (MEFs) are from Jay L. Hess's lab, University of Michigan. All cell lines were maintained in Dulbecco's Modification of Eagle's Medium, McCoy's 5a medium or advanced RPMI medium 1640 (Invitrogen) with 10% FBS (Invitrogen). Infected cell lines were maintained under 1 μg/mL of puromycin (MP Biomedicals) for selection.
siRNA Screening
A375 cell line with integrated HSP70 promoter-driven luciferase reporter activated by HSP90 inhibitor treatment was established. To perform a high-throughput genome-wide siRNA screen, the full siRNA library was stamped out in 384 well plates, as well as HSF1 siRNA and negative controls. RNAiMAX was added to each well and further be incubated. Then, cancer cells with HSP70 promoter-driven luciferase reporter were plated and incubated for 72 h, then HSP990 was added and incubated for 6 h. Finally, Bright-Glo (BG) was added to measure luminescence of the HSP70 reporter. In the 2^{nd} round screen, siRNA screen data was analyzed by both BG and CellTiter-Glo (CTG) assays; the latter will measure overall cell viability. 1) Data was normalized and exported to a spotfire file for viewing. 2) An average by siRNA replicate was calculated for each assay. 3) Following this, differences between the BG and CTG scores for each siRNA average were taken. 4) These differences were averaged for each Gene ID and then sorted by delta (the greatest difference between BG and CTG should then be the strongest hits since the top hits that affecting BG signal without affecting CTG were searched). The counter screening assays, such as examining the endogenous HSP70 gene expression after knockdown of potential HSF1-modulators selected from above screen and examining potential HSF1-modulators genes knockdown, were also performed.

### Short Hairpin RNA Constructs

Control short hairpin RNA (shRNA), GGATAATGGTGATTGAGATGG, MLL1 shRNA#1, GCACTGTTAAACATTCCACTT, and MLL1 shRNA#2, CGCCTAAAGCAGCTCTCATTT, were cloned into the inducible pLKO-Tet-On puromycin vector.

### Lentivirus and Infection

Lentiviral supernatants were generated according to our previously established protocol. A total of 100 μL of lentivirus was used to infect 300,000 cancer cells in a six-well plate, in 8 μg/mL polybrene (Chemicon). Medium was replaced and after 24 h, cells were selected by puromycin (MP Biomedicals) and expanded. Induction of shRNA was obtained by addition of 100 ng/mL Doxycyclineycycline (Clontech) to the medium.

### RNA Extraction and Quantitative Reverse Transcription-PCR

Total RNA was isolated using the RNeasyMini kit (Qiagen). ABI taqman gene expression assays include HSP70, BAG3, HSC70, HSP27, HSF1 and MLL1. VICMGB primers/probe sets (Applied Biosystems) were used in each reaction to coamplify the B2M transcripts. All experiments were performed in either duplicate or triplicate and normalize to B2M levels as indicated.

### Chromatin Immunoprecipitation (ChIP) Assay

ChIP assay was carried out according to the manufacturers protocol (chromatin immunoprecipitation assay kit, catalog no. 17-295, Upstate Biotechnology Inc., Lake Placid, N.Y.). Immune complexes were prepared using anti-HSF1 (Cell Signaling, 4356) antibody, anti-MLL1 (Bethyl Laboratories, A300-086A), anti-H3K4Me2 (Thermo scientific, MA511196), anti-H3K4Me3 (Thermo scientific, MA511199), and anti-H4K16Ac (Millipore, 07-329). The supernatant of immunoprecipitation reaction carried out in the absence of antibody served as the total input DNA control. PCR was carried out with 10 μl of each sample using the following primers: HSP70 promoter, 5′-GGCGAAACCCCTGGAATATTCCCGA-3′ and 5′-AGCCTTGGGACAACGGGAG-3′; BAG3 promoter, 5′-GTCCCCTCCTTACAAGGAAA-3′ and 5′-CAATTGCACTTGTAACCTG-3; MEIS1 promoter, 5′-CGGCGTTGATTCCCAATTTATTTCA-3′ and 5′-CACACAAACGCAGGCAGTAG-3′. This was followed by analysis on 2% agarose gels.

### Gene Profiling

RNA was isolated using the Qiagen RNeasy mini kit. Generation of labeled cDNA and hybridization to HG-U133 Plus2 arrays (Affymetrix) were performed as previously described (45).

### Western Blotting

Western blottings were performed as follows: total tumor lysates were separated by SDS/PAGE and electrotransferred to nitrocellulose membrane (Invitrogen). Membranes were blocked in PBS and 0.1% (vol/vol) Tween-20 (PBS-T) and 4% (wt/vol) nonfat dry milk (Bio-Rad) for 1 h on a shaker at room temperature. Primary antibodies were added to the blocking solution at 1:1,000 (HSF1; Cell signaling, 4356), 1:1,000 (HSP70; Cell signaling, 4876), 1:1,000 (p-ERK; Cell signaling, 4370), 1:1,000 (ERK; Cell signaling, 4695), 1:1,000 (HER2; Cell signaling, 4290), 1:1,000 (BRAF; Cell signaling, 9433), 1:1,000 (cleaved PARP; Cell signaling, 5625), and 1:10,000 (GAPDH; Cell Signaling Technology, 2118S) dilutions and incubated overnight and a rocker at 4° C. Immunoblottings were washed three times, 5 min each with PBS-T, and secondary antibody was added at 1:10,000 dilution into PBS-T milk for 1 h on a shaker at room temperature. After several washes, enhanced chemiluminescence (ECL) reactions were performed according to manufacturer's recommendations (SuperSignal West Dura Extended Duration Substrate; Thermo Scientific).

### Tumor Xenografts

Mice were maintained and handled in accordance with Novartis Biomedical Research Animal Care and Use Committee protocols and regulations. A375 with Tet-inducible shRNA against MLL1 were cultured in DMEM supplemented with 10% Tet-approved FBS. Mice (6-8 wk old, n=8) were inoculated s.c. with 1×10⁶ cells in the right dorsal axillary region. Tumor volume was measured by calipering in two dimensions and calculated as (length×width2)/2. Drug treatment started 11 d after implant when average tumor volume was 200 mm³. Animals received vehicle (5% dextrose, 10 mL/kg, orally, qw) or HSP990 (10 mg/kg, orally, qw) for the duration of the study. At termination of the study, tumor tissues were excised and snap frozen in liquid nitrogen for immunoblotting analyses of biomarkers. Data were expressed as mean±SEM, and differences were considered statistically significant at P<0.05 by Student t test.

### Authors' Contributions

YC and WZ designed the experiments. YC, JC, AL, LB, DR, RG and MM performed the experiments. SJ, JY and JK analyzed the data. FC, PZ, FS, RP and DP helped with the experiments. YC and WZ wrote the paper.

### Figure Legends:

### Table 1: IC95 of AUY922 Among Eight Human Leukemia Cells

Eight human leukemia cells with or without MLL1 translocation were treated with AUY922 for 72 h and cell proliferation rate were measured by CellTiter-Glo. IC95 were used to estimate the cell response to HSP90 inhibition.
Supplementary Table. S1: 35 Genes were Identified as Modulators of Cell Response to HSP90 Inhibition by siRNA Screening

1. A method of selectively treating a subject having cancer, including selectively administering a therapeutically effective amount of (5-(2,4-Dihydroxy-5-isopropyl-phenyl)-4-(4-morpholin-4-ylmethyl-phenyl)-isoxazole-3-carboxylic acid ethylamide, or a pharmaceutically acceptable salt thereof, to the subject on the basis of the subject having reduced levels of MLL1

2. A method according to claim 1 further comprising:
a) assaying a biological sample from the subject for the level of MLL1; and
b) selectively administering a therapeutically effective amount of (5-(2,4-Dihydroxy-5-isopropyl-phenyl)-4-(4-morpholin-4-ylmethyl-phenyl)-isoxazole-3-carboxylic acid ethylamide or a pharmaceutically acceptable salt thereof, to the subject on the basis that the sample has reduced levels of MLL1.

3. (canceled)

4. A method according to claim 2 further comprising:
a) assaying a biological sample from the subject for the levels of MLL1;
b) thereafter selecting the subject for treatment with (5-(2,4-Dihydroxy-5-isopropyl-phenyl)-4-(4-morpholin-4-ylmethyl-phenyl)-isoxazole-3-carboxylic acid ethylamide (AUY922), or a pharmaceutically acceptable salt thereof, on the basis that the subject has reduced levels of MLL1; and
c) thereafter administering (5-(2,4-Dihydroxy-5-isopropyl-phenyl)-4-(4-morpholin-4-ylmethyl-phenyl)-isoxazole-3-carboxylic acid ethylamide or a pharmaceutically acceptable salt thereof to the subject on the basis that the subject has reduced levels of MLL1.

5-7. (canceled)

8. A method of genotyping an individual including detecting a genetic variant that results in an amino acid variant at position 859 of the encoded catalytic p110α subunit of PI3K, wherein a lack of variant at position 859 indicates that (5-(2,4-Dihydroxy-5-isopropyl-phenyl)-4-(4-morpholin-4-ylmethyl-phenyl)-isoxazole-3-carboxylic acid ethylamide should be administered to the individual.

9. (canceled)

10. An HSP90 inhibitor compound (5-(2,4-Dihydroxy-5-isopropyl-phenyl)-4-(4-morpholin-4-ylmethyl-phenyl)-isoxazole-3-carboxylic acid ethylamide or a pharmaceutically acceptable salt thereof, for use in treating cancer, characterized in that a therapeutically effective amount of said compound or its pharmaceutically acceptable salt is administered to an individual on the basis of the individual having reduced MLL1 levels compared to a control at one or more of the following positions:
(a) 146982000-146984500 on chromosome X of an FMR1 genomic locus;
(b) 146991600-146993600 on chromosome X of an FMR1 genomic locus;
(c) 146994300-147005500 on chromosome X of an FMR1 genomic locus; or
(d) 147023800-147027400 on chromosome X of an FMR1 genomic locus.

11. (canceled)

12. The method according to claim 1, wherein the cancer is selected from the group consisting of glioblastoma; melanoma; ovarian cancer; breast cancer; lung cancer; non-small-cell lung cancer (NSCLC); endometrial cancer, prostate cancer: colon cancer; and myeloma.

13. The method according to claim 1, wherein the sample is a tumor sample.

14. The method of claim 13, wherein the tumor sample is a fresh frozen sample or a parrafin embedded tissue sample.

15. The method of according to claim 14, wherein the detecting can be performed by immunoassays, immunohistochemistry, ELISA, flow cytometry, Western blot, HPLC, and mass spectrometry.

16. The method according to claim 15, wherein the presence or absence of a mutation in a nucleic acid molecule encoding the catalytic p110α subunit of the PI3K can be detected by a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis or Southernblot.

17. The method of claim 15, wherein said detecting step comprises sequencing the catalytic p110α subunit gene of PI3K or a portion thereof.

18. (canceled)

19. A kit for determining if a tumor is responsive for treatment with the HSP90 inhibitor compound (5-(2,4-Dihydroxy-5-isopropyl-phenyl)-4-(4-morpholin-4-ylmethyl-phenyl)-isoxazole-3-carboxylic acid ethylamide or a pharmaceutically acceptable salt thereof comprising providing one or more probes or primers for detecting the presence of a mutation at the PI3K gene locus (nucleic acid 2575-2577 of SEQ ID NO:2) and instructions for use.

20. A kit according to claim 19 for predicting whether a subject with cancer would benefit from treatment with the HSP90 inhibitor compound (5-(2,4-Dihydroxy-5-isopropyl-phenyl)-4-(4-morpholin-4-ylmethyl-phenyl)-isoxazole-3-carboxylic acid ethylamide or a pharmaceutically acceptable salt thereof, the kit comprising:
d) a plurality of agents for determining for the presence of a mutation that encodes a variant at position 859 of the catalytic p110α subunit of PI3K; and
e) instructions for use.

21. (canceled)
